(19) United States
(12) Patent Application Publication
(10) Pub. No.: US 2022/0148453 A1
(43) Pub. Date: May 12, 2022

(54) VISION-BASED REHABILITATION TRAINING SYSTEM BASED ON 3D HUMAN POSE ESTIMATION USING MULTI-VIEW IMAGES

(71) Applicant: TENCENT AMERICA LLC, Palo Alto, CA (US)

(72) Inventors: Shih-Yao LIN, Palo Alto, CA (US); Hui TANG, Mountain View, CA (US); Chao HUANG, Palo Alto, CA (US); Lianyi HAN, Palo Alto, CA (US); Zhimin HUO, Pittsford, NY (US); Wei FAN, New York, NY (US)

(73) Assignee: TENCENT AMERICA LLC, Palo Alto, CA (US)

(21) Appl. No.: 17/096,256

(22) Filed: Nov. 12, 2020

(51) Int. Cl. G09B 19/00; G06K 9/00; G06T 7/70; G06T 13/40; H04N 5/247; G06T 7/20; G09B 5/06; A61B 5/11; A61B 5/00

(52) U.S. Cl. CPC G09B 19/003; G06K 9/00342; G06T 7/70; G06T 13/40; H04N 5/247; G06T 7/20; G06T 2207/30196; G09B 5/065; A61B 5/1127; A61B 5/744; A61B 5/7405; A61B 5/4528; G06T 2207/10016; G06K 9/00711

(57) ABSTRACT

Systems and methods for marker-free motion capture are provided. A method includes: obtaining a plurality of videos of a body of a person; estimating a three dimensional (3D) pose of the person based on the plurality of videos without depending on any marker on the person, the estimating including obtaining a set of 3D body joints; obtaining an animation of motion of the set of 3D body joints that corresponds to motion of the person during a time period; performing an analysis of the motion of the set of 3D body joints; and indicating a rehabilitation evaluation result of the analysis or a rehabilitation training suggestion, based on the analysis, via a display or a speaker.

### FIELD

Embodiments of the present disclosure are directed to rehabilitation systems and, more particularly, marker-free motion capture systems.

### BACKGROUND

Traditional rehabilitation systems require patients to wear specific sensors on their bodies. However, such sensor-based systems cause inconvenience to the patients. Some of recent works estimate hand poses with a depth sensor for hand recovery training. However, using a special sensor in a system limits generalization. Furthermore, the traditional devices are usually expensive.

### SUMMARY

Embodiments of the present disclosure may solve the above problems and/or other problems.

Embodiments of the present disclosure may provide a marker-free motion capture system, using vision-based technology, which can estimate three dimensional (3D) human body poses based on multi-view images captured by low-cost commercial cameras (e.g. three cameras).

Embodiments of the present disclosure may provide a multi-view 3D human pose estimation for rehabilitation training of, for example, movement disorders. Based on the multi-view images captured by low-cost cameras, deep learning models of embodiments of the present disclosure can calculate precise 3D human poses. Embodiments of the present disclosure may not only obtain 3D body joints, but may also provide evaluation results of patients' motion and rehabilitation suggestions. Accordingly, rehabilitation training evaluation and guidance can be realized without the assistance of doctors in the process.

Embodiments of the present disclosure may include modules for representing animation for the patients to monitor their motions and poses, and to improve their training. Moreover, embodiments of the present disclosure may include evaluation indicators and may provide suggestions to help the patients improve their rehabilitation. According to embodiments, 3D human pose estimation techniques may be leveraged for rehabilitation training, which has not been accomplished by related art.

Embodiments of the present disclosure may provide a vision-based, marker-free, motion capture system for rehabilitation training, which avoids limitations of traditional motion capture systems and has not been accomplished by related art. Embodiments of the present disclosure may include combinations of video and voice guidance, as a part of contactless rehabilitation training evaluation and guidance. Embodiments of the present disclosure may estimate a 3D human pose based on deep learning technology with multi-view images in various perspectives. The information of the multi-view images may assist the deep learning technology to accurately infer the 3D human pose.

According to one or more embodiments, a method performed by at least one processor is provided. The method includes: obtaining a plurality of videos of a body of a person, the plurality of videos including a first video of the person from a first perspective that is captured by a first camera during a time period, and a second video of the person from a second perspective, different from the first perspective, that is captured by a second camera during the time period; estimating a three dimensional (3D) pose of the person based on the plurality of videos without depending on any marker on the person, the estimating including obtaining a set of 3D body joints; obtaining an animation of motion of the set of 3D body joints that corresponds to motion of the person during the time period; performing an analysis of the motion of the set of 3D body joints; and indicating a rehabilitation evaluation result of the analysis or a rehabilitation training suggestion, based on the analysis, via a display or a speaker.

According to an embodiment, the performing the analysis includes calculating at least one rehabilitation evaluation indicator based on the motion of the set of 3D body joints.

According to an embodiment, the performing the analysis further includes selecting the at least one rehabilitation evaluation indicator to be calculated based on an input from a user.

According to an embodiment, the method further includes displaying the animation of the motion of the set of 3D body joints.

According to an embodiment, the animation of the motion of the set of 3D body joints is displayed in real-time with respect to the motion of the person during the time period.

According to an embodiment, the animation includes images of the body of the person combined with the set of 3D body joints.

According to an embodiment, the plurality of videos, that are obtained, further includes a third video of the person from a third perspective, different from the first perspective and the second perspective, that is captured by a third camera during the time period.

According to an embodiment, the first perspective is a left side view of the person, the second perspective is a front view of the person, and the third perspective is a right side view of the person.

According to an embodiment, the second camera captures the second video at a higher height than a height at which the first camera captures the first video and a height at which the third camera captures the third video.

According to an embodiment, the height at which the first camera captures the first video and the height at which the third camera captures the third video are a same height.

According to one or more embodiments, a system is provided. The system includes: a plurality of cameras, the plurality of cameras configured to each obtain a respective video from among a plurality of videos of a body of a person. The plurality of cameras include: a first camera configured to obtain a first video, from among the plurality of videos, of the person from a first perspective during a time period, and a second camera configured to capture a second video, from among the plurality of videos, of the person from a second perspective, different from the first perspective, during the time period. The system further includes a display or a speaker; at least one processor; and memory including computer code. The computer code includes: first code configured to cause the at least one processor to estimate a three dimensional (3D) pose of the person by obtaining a set of 3D body joints, based on the plurality of videos without depending on any marker on the person; second code configured to cause the at least one processor to obtain an animation of motion of the set of 3D body joints that corresponds to motion of the person during the time period; third code configured to cause the at least one processor to perform an analysis of the motion of the set of 3D body joints; and fourth code configured to cause the at least one processor to indicate a rehabilitation evaluation result of the analysis or a rehabilitation training suggestion, based on the analysis, via the display or the speaker.

According to an embodiment, the third code is configured to cause the at least one processor to perform the analysis by calculating at least one rehabilitation evaluation indicator based on the motion of the set of 3D body joints.

According to an embodiment, the third code is further configured to cause the at least one processor to select the at least one rehabilitation evaluation indicator to be calculated based on an input from a user.

According to an embodiment, the system includes the display, and the second code is further configured to cause the at least one processor to cause the display to display the animation of the motion of the set of 3D body joints.

According to an embodiment, the second code is configured to cause the at least one processor to cause the display to display the animation in real-time with respect to the motion of the person during the time period.

According to an embodiment, the animation includes images of the body of the person combined with the set of 3D body joints.

According to an embodiment, the plurality of cameras further includes a third camera that is configured to obtain a third video of the person from a third perspective, different from the first perspective and the second perspective, during the time period.

According to an embodiment, the first perspective is a left side view of the person, the second perspective is a front view of the person, and the third perspective is a right side view of the person.

According to an embodiment, the second camera is at a higher height than a height of the first camera and a height of the third camera.

According to one or more embodiments, a non-transitory computer-readable medium storing computer code is provided. The computer code is configured to, when executed by at least one processor, cause the at least one processor to: estimate a three dimensional (3D) pose of a person by obtaining a set of 3D body joints based on a plurality of videos of a body of the person without depending on any marker on the person; obtain an animation of motion of the set of 3D body joints that corresponds to motion of the person during a time period; perform an analysis of the motion of the set of 3D body joints; and indicate a rehabilitation evaluation result of the analysis or a rehabilitation training suggestion, based on the analysis, via a display or a speaker. The plurality of videos include a first video of the person from a first perspective that is captured by a first camera during the time period, and a second video of the person from a second perspective, different from the first perspective, that is captured by a second camera during the time period.

### BRIEF DESCRIPTION OF THE DRAWINGS

Further features, the nature, and various advantages of the disclosed subject matter will be more apparent from the following detailed description and the accompanying drawings in which:

FIG. 1 is schematic illustration of a rehabilitation training system according to embodiments.

FIG. 2 is a block diagram of processes according to embodiments of the present disclosure.

FIG. 3 is a schematic illustration of computer code according to embodiments of the present disclosure.

FIG. 4 is a perspective view illustration of a camera configuration according to embodiments of the present disclosure.

FIG. 5 is an example illustration of a patient's pose that is represented by 3D body joints, according to embodiments of the present disclosure

FIG. 6 is a block diagram of a process according to embodiments of the present disclosure.

FIG. 7A is an example illustration of a portion of a displayed animation according to embodiments of the present disclosure.

FIG. 7B is an example illustration of a portion of a displayed animation according to embodiments of the present disclosure.

FIG. 8 is a schematic illustration of a computer system according to embodiments of the present disclosure.

### DETAILED DESCRIPTION

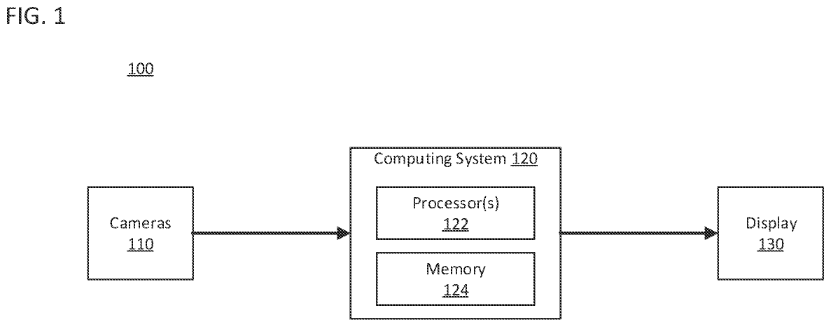

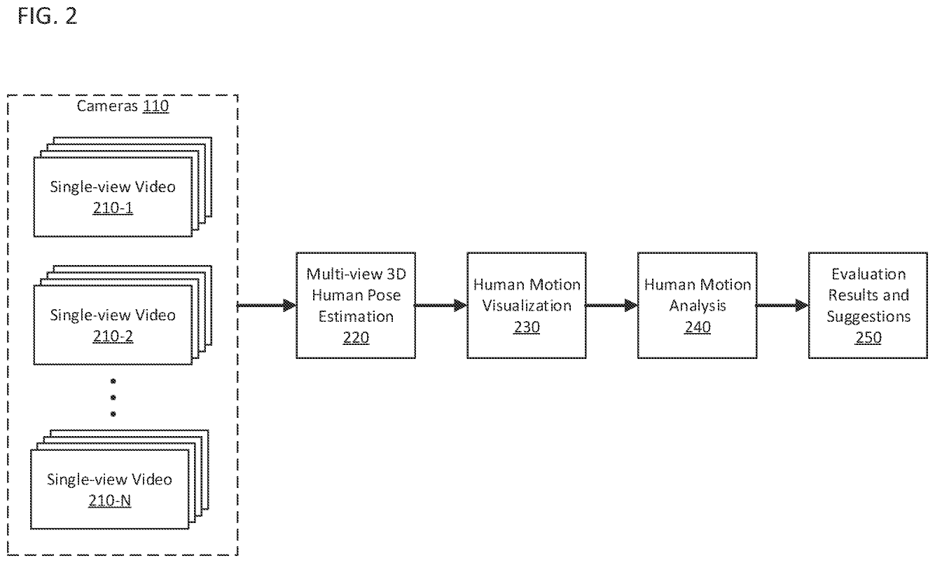

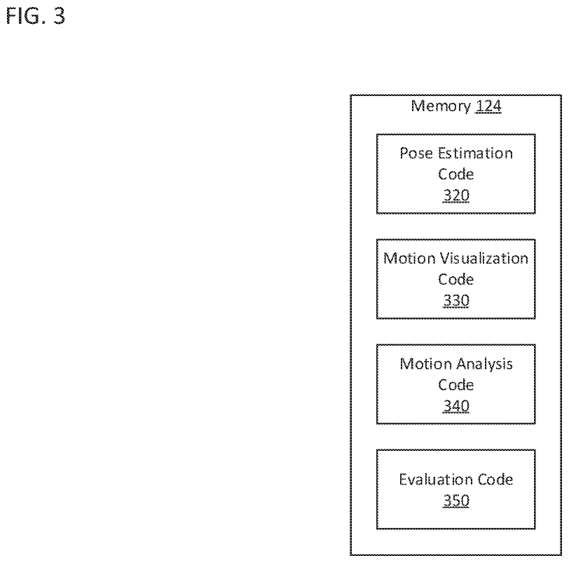

According to embodiments, with reference to FIG. 1, a rehabilitation training system 100 is provided. The rehabilitation training system 100 may include, for example, cameras 110, a computing system 120, and a display 130. The cameras 110 may include any number of cameras. For example, according to embodiments, the cameras 110 may include two or three cameras. The cameras 110 may be configured to obtain video data, and transmit the video data via a wired or wireless connection to the computing system 120. The computing system 120 may include at least one processor 122 and memory storing computer code. The computer code may be configured to, when executed by the at least one processor 122, cause the at least one processor 122 to perform the processes of the computing system 120 such as those described below with respect to FIG. 2. An example diagram of the computer code is illustrated in FIG. 3. The computing system 120 may also include, or be connected to, the display 130, and may be configured to cause the display 130 to display results of the processes of the computing system 120. The computing system 120 may be connected to the display 130 via a wired or wireless connection.

With reference to FIGS. 2-3, processes performed by the computing system 120 is described below. With reference to FIG. 2, the computing system 120 may perform the processes of multi-view 3D human pose estimation (220), human motion visualization (230), human motion analysis (240), and provision of evaluation results and suggestions (250). With reference to FIG. 3, such processes may be respectively caused to be executed by the at least one processor 122 of the computing system 120 by pose estimation code 320, motion visualization code 330, motion analysis code 340, and evaluation code 350 included in the memory 124.

The computing system 120 may receive video data from the cameras 110 as inputs to the multi-view 3D human pose estimation (220). For example, each of the cameras 110 may provide, to the computing system 120, a single-view video (e.g. single-view video 210-1, 210-2, . . . , 210-N) that each include images of a patient from a respective perspective. In other words, each of the cameras 110 may capture a patient's pose and motion from a respective direction in a respective single-view video (e.g. single-view video 210-1, 210-2, , 210-N), which are then obtained by the computing system 120 from the cameras 110.

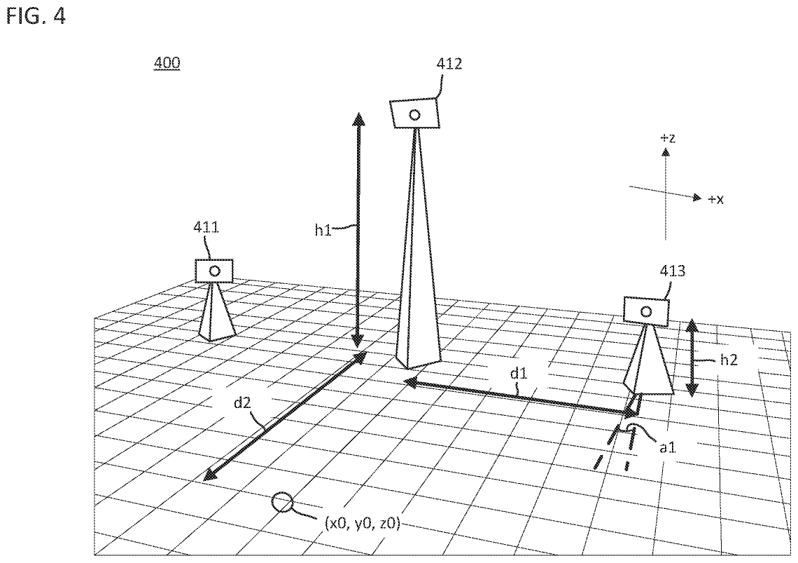

As an example, with reference to FIG. 4, the cameras 110 of the rehabilitation training system 100 may include a first camera 411, a second camera 412, and a third camera 413 in a configuration 400. In the configuration 400, the first camera 411, the second camera 412, and the third camera 413 may be provided at respective positions to capture respective viewpoints of a patient that starts at a position (x0, y0, z0). With reference to FIG. 4, an x-direction may be along an x-axis that extends in a left-right direction relative to FIG. 4 (+x direction being towards the right of FIG. 4), a y-direction may be along a y-axis that extends into or out of FIG. 4 (+y direction being into FIG. 4), and a z-direction may be along a z-axis that extends in an up-down direction relative to FIG. 4 (+z direction being towards the top of FIG. 4). The second camera 412 may be at a same or similar x-position as the position (x0, y0, z0) at which the patient starts, and may be at a height h1 above the position (x0, y0, z0) (e.g. above the ground height) in the +z direction. The first camera 411 may be in the −x direction at the distance d1 relative to the position (x0, y0, z0) and/or the second camera 412, and the third camera 413 may be in the +x direction at the distance d1 relative to the position (x0, y0, z0) and/or the second camera 412. The first camera 411 and the third camera 413 may be at a same height h2 above the position (x0, y0, z0) (e.g. above the ground height) in the +z direction. The first camera 411, the second camera 412, and the third camera 413 may each be at a same y-position (e.g. a +y position). Each of the first camera 411, the second camera 412, and the third camera 413 may have a respective view angle al that is angled with respect to at least one axis towards the position (x0, y0, z0). For example, as indicated in FIG. 4, the view angle al of the third camera 413 may at least be angled from the y-axis in the −x direction. Additionally, the view angle of the first camera 411 may at least be angled from the y-axis in the +x direction, and the view angle of the second camera 412 may at least be angled from the y-axis in the −z direction. According to the configuration 400, the first camera 411 may be configured to capture a left side perspective of the patient's body, the second camera 412 may be configured to capture an upper/front perspective of the patient's body, and the third camera 413 may be configured to capture a right side perspective of the patient's body.

While FIG. 4 illustrates a configuration 400, other camera configurations with a different number of cameras 110, camera positions, and/or camera view angles may be implemented in embodiments of the present disclosure.

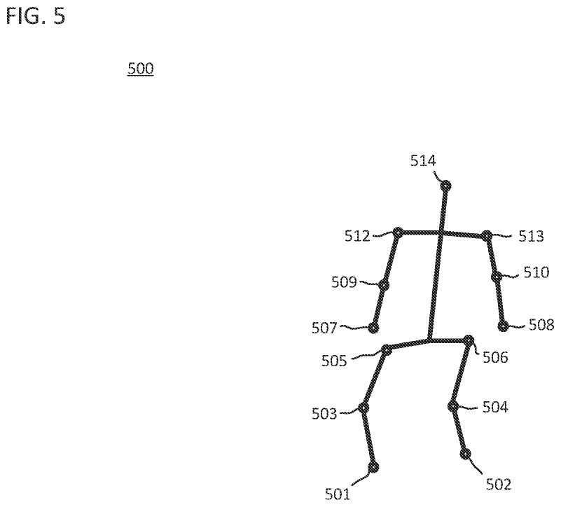

As described above, the cameras 110 may be provided in various positions and with various view angles to capture various perspectives of a patient, and video data from the cameras 110 may be input to the computing system 120 to perform a multi-view 3D human pose estimation (220). The multi-view 3D human pose estimation (220) may be a process in which the computing system 120 uses the video data from the cameras 110 to infer a pose(s) of the patient and represent the pose(s) as a set of 3D joint locations. An example of a patient's pose represented by 3D body joints is shown in FIG. 5. As shown in FIG. 5, a pose 500 may be represented with various body joints including, for example, a right foot joint 501, a left foot joint 502, a right knee joint 503, a left knee joint 504, a right hip joint 505, a left hip joint 506, a right hand joint 507, a left hand joint 508, a right elbow joint 509, a left elbow joint 510, a right shoulder joint 512, a left shoulder joint 513, and a head joint 514.

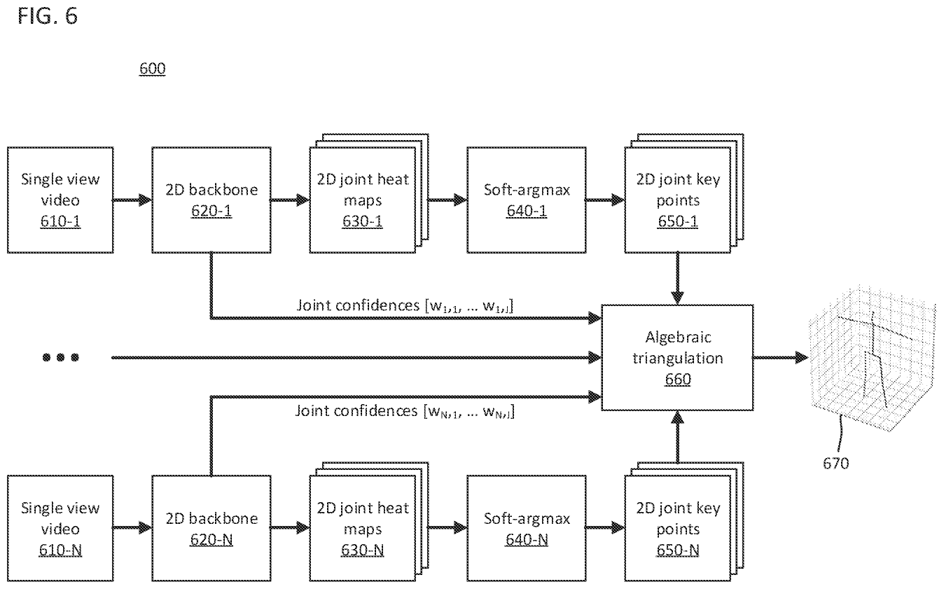

According to embodiments, with reference to FIG. 6, the multi-view 3D human pose estimation (220) may be performed by the computing system 120 using a process 600. The process 600 may be implemented by an end-to-end deep neural network (DNN) model.

The process 600 may be a two stage approach in which the 2D coordinates of body joints are estimated in each single camera view and, then, triangular and linear regression is used to take multi-view information into account to infer a 3D human pose.

For example, with reference to FIG. 6, the process 600 may include obtaining, from each of the cameras 110, a respective single view video (e.g. single view videos 610-1, . . . , 610-N). Based on each single view video 610-1, . . . , 610-N, a respective 2D backbone 620-1, . . . , 620-N may be obtained. Based on each 2D backbone 620-1, . . . , 620-N, a respective set of 2D joint heat maps 630-1, , 630-N may be obtained. Each set of 2D joint heat maps 630-1, . . . , 630-N, may be input into a respective soft-argmax function 640-1, . . . , 640-N to obtain a respective set of 2D joint key points 650-1, . . . , 650-N. Following, algebraic triangulation 660 may be performed using all of the sets of the 2D joint key points 650-1, . . . , 650-N, and using joint confidences obtained based on each 2D backbone 620-1, . . . , 620-N, to obtain the set of 3D body joint locations 670, which are a set of 3D estimated body joints.

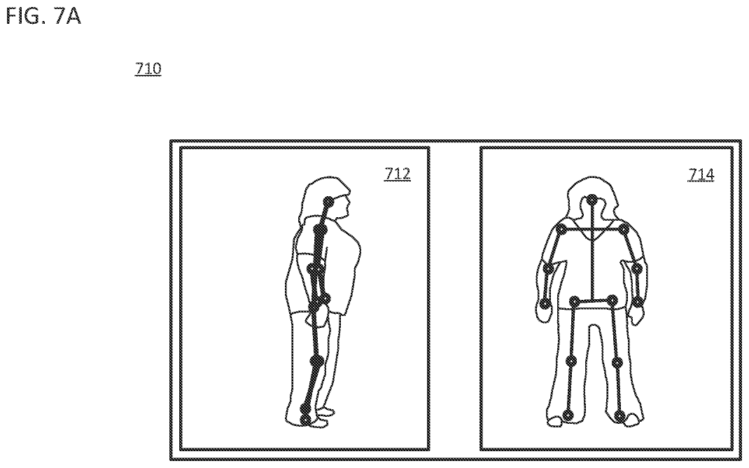

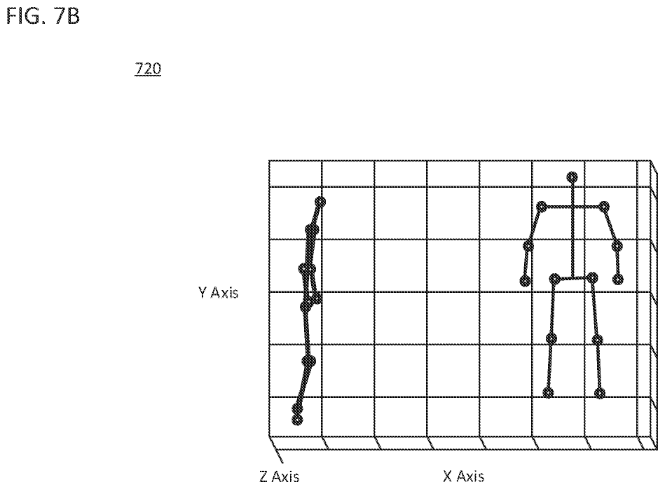

With reference to FIGS. 7A-B, the computing system 120 may be configured to perform the human motion visualization (230) process in which estimated 3D human motion for a patient is represented based on the set of 3D estimated body joints (e.g. the set of 3D body joint locations 670). The human motion visualization (230) process may include removing noise caused by failure pose estimation, and generating real-time animation.

For example, as shown in FIG. 7A, the computing system 120 may be configured to combine video images of a patient with the set of 3D estimated body joints (e.g. set of 3D body joint locations 670) of the patient, and display the combination as an animation 710. According to embodiments, the animation 710 may simultaneously include multiple perspective video images of the patient combined with the set of 3D estimated body joints. As an example, the animation 710 is shown with a right perspective video 712 of the patient and a front perspective video 714 of the patient. However, the number of videos and types of perspectives may vary in the animation 710.

Also, as shown in FIG. 7B, the computing system 120 may be configured to generate an animation 720 which is similar to the animation 710, except the set of 3D estimated body joints in shown in multiple perspectives simultaneously, without video images of the patient being shown therewith.

According to embodiments, the animation 710 and the animation 720 may be displayed simultaneously. According to embodiments, the animation 710 and the animation 720 may be real-time animations. According to embodiments, the multiple perspective video images of the patient, that are combined with the set of 3D estimated body joints, may be obtained from two or more of the single-view videos 210-1, ... 210-N (refer to FIG. 2). According to embodiments, the computing system 120 may cause the animation 710 and or the animation 720 to be displayed on the display 130 (refer to FIG. 1).

By displaying animations in accordance with embodiments of the present disclosure, patients may better monitor their movements and postures, which can help them to understand how they perform in the rehabilitation training.

The computing system 120 may also be configured to perform the human motion analysis (240) process, in which a user may set different evaluation indicators according to rehabilitation training types. The computing system 120 may then calculate the indicators based on estimated 3D human motion obtained from the multi-view 3D human pose estimation (220) process and the human motion visualization (230) process. The estimated 3D human motion may refer to the animated motion of the set of 3D estimated body joints (e.g. the set of 3D body joint locations 670) (refer to FIGS. 6-7B). An example of a rehabilitation training type is rehabilitation training of walking movement. The indicators of rehabilitation training of walking movement may include the patient's walking speed, the height of the patient's leg, walking stability, and the amplitude and frequency of the patient's arm swing. According to embodiments, the computing system 120 may automatically determine the indicators to be calculated based on a user selecting a rehabilitation training type using an input device (e.g. a mouse, keyboard, touch screen, microphone, etc.) that is connected to the computing system 120. According to embodiments, a user may manually select the indicators to be calculated using the input device, and the computing system 120 may be configured to perform the calculations based on the selection(s).

Following the human motion analysis (240) process, the computing system 120 may be configured to perform the evaluation results and suggestions (250) process. That is, for example, evaluation results may be determined by the computing system 120 based on a result(s) of the human motion analysis (240) process, and training suggestions (with or without the evaluation results) may be provided (e.g. displayed on the display 130 or output by a speaker) to the patient based on the evaluation results. As an example. when an evaluation result is that a patient's walking movement is determined to be too slow due to arm swing amplitude being too low, the computing system 120 may provide a training suggestion that the patient should strengthen his or her arm swing. According to embodiments, the results and suggestions (250) process that is performed by the computing system 120 may include calculating and providing (e.g. displaying on the display 130 or outputting by a speaker) a final evaluation score to the patient based on the result(s) of the human motion analysis (240) process.

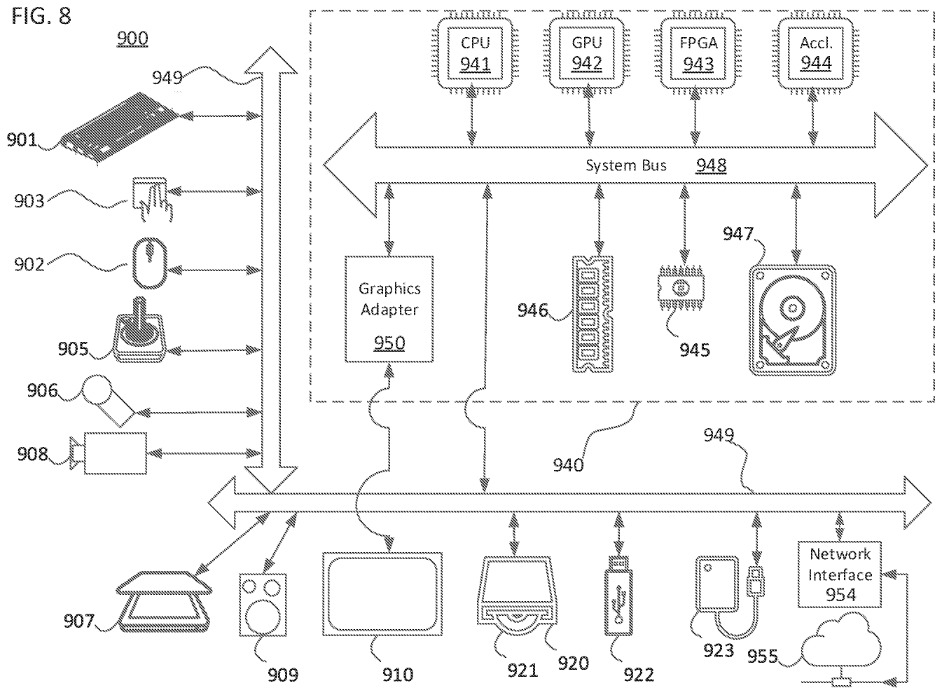

The processes of the present disclosure, described above, can be implemented as computer software using computer-readable instructions and physically stored in one or more computer-readable media. For example, FIG. 8 shows a computer system 900 suitable for implementing the computing system 120 of the disclosed subject matter.

The computer software can be coded using any suitable machine code or computer language, that may be subject to assembly, compilation, linking, or like mechanisms to create code including instructions that can be executed directly, or through interpretation, micro-code execution, and the like, by computer central processing units (CPUs), Graphics Processing Units (GPUs), and the like.

The instructions can be executed on various types of computers or components thereof, including, for example, personal computers, tablet computers, servers, smartphones, gaming devices, intemet of things devices, and the like.

The components shown in FIG. 8 for computer system 900 are exemplary in nature and are not intended to suggest any limitation as to the scope of use or functionality of the computer software implementing embodiments of the present disclosure. Neither should the configuration of components be interpreted as having any dependency or requirement relating to any one or combination of components illustrated in the exemplary embodiment of a computer system 900.

Computer system 900 may include certain human interface input devices. Such a human interface input device may be responsive to input by one or more human users through, for example, tactile input (such as: keystrokes, swipes, data glove movements), audio input (such as: voice, clapping), visual input (such as: gestures), olfactory input (not depicted). The human interface devices can also be used to capture certain media not necessarily directly related to conscious input by a human, such as audio (such as: speech, music, ambient sound), images (such as: scanned images, photographic images obtain from a still image camera), video (such as two-dimensional video, three-dimensional video including stereoscopic video).

Input human interface devices may include one or more of (only one of each depicted): keyboard 901, mouse 902, trackpad 903, touch-screen 910, joystick 905, microphone 906, scanner 907, and camera 908.

Computer system 900 may also include certain human interface output devices. Such human interface output devices may be stimulating the senses of one or more human users through, for example, tactile output, sound, light, and smell/taste. Such human interface output devices may include tactile output devices for example tactile feedback by the touch-screen 910, data-glove, or joystick 905, but there can also be tactile feedback devices that do not serve as input devices. For example, such devices may be audio output devices (such as: speakers 909), headphones (not depicted)), visual output devices (such as screens 910 to include CRT screens, LCD screens, plasma screens, OLED screens, each with or without touch-screen input capability, each with or without tactile feedback capability—some of which may be capable to output two dimensional visual output or more than three dimensional output through means such as stereographic output; virtual-reality glasses (not depicted), holographic displays and smoke tanks (not depicted), and printers (not depicted).

Computer system 900 can also include human accessible storage devices and their associated media such as optical media including CD/DVD ROM/RW 920 with CD/DVD or the like media 921, thumb-drive 922, removable hard drive or solid state drive 923, legacy magnetic media such as tape and floppy disc (not depicted), specialized ROM/ASIC/PLD based devices such as security dongles (not depicted), and the like.

Those skilled in the art should also understand that term “computer readable media” as used in connection with the presently disclosed subject matter does not encompass transmission media, carrier waves, or other transitory signals.

Computer system 900 can also include interface to one or more communication networks. Networks can for example be wireless, wireline, optical. Networks can further be local, wide-area, metropolitan, vehicular and industrial, real-time, delay-tolerant, and so on. Examples of networks include local area networks such as Ethernet, wireless LANs, cellular networks to include GSM, 3G, 4G, 5G, LTE and the like, TV wireline or wireless wide area digital networks to include cable TV, satellite TV, and terrestrial broadcast TV, vehicular and industrial to include CANBus, and so forth. Certain networks commonly require external network interface adapters that attached to certain general purpose data ports or peripheral buses 949 (such as, for example USB ports of the computer system 900; others are commonly integrated into the core of the computer system 900 by attachment to a system bus as described below (for example Ethernet interface into a PC computer system or cellular network interface into a smartphone computer system). Using any of these networks, computer system 900 can communicate with other entities. Such communication can be uni-directional, receive only (for example, broadcast TV), uni-directional send-only (for example CANbus to certain CANbus devices), or bi-directional, for example to other computer systems using local or wide area digital networks. Such communication can include communication to a cloud computing environment 955. Certain protocols and protocol stacks can be used on each of those networks and network interfaces as described above.

Aforementioned human interface devices, human-accessible storage devices, and network interfaces 954 can be attached to a core 940 of the computer system 900.

The core 940 can include one or more Central Processing Units (CPU) 941, Graphics Processing Units (GPU) 942, specialized programmable processing units in the form of Field Programmable Gate Areas (FPGA) 943, hardware accelerators 944 for certain tasks, and so forth. These devices, along with Read-only memory (ROM) 945, Random-access memory (RAM) 946, internal mass storage such as internal non-user accessible hard drives, SSDs, and the like, may be connected through a system bus 948. In some computer systems, the system bus 948 can be accessible in the form of one or more physical plugs to enable extensions by additional CPUs, GPU, and the like. The peripheral devices can be attached either directly to the core's system bus 948, or through a peripheral bus 949. Architectures for a peripheral bus include PCI, USB, and the like. A graphics adapter 950 may be included in the core 940.

CPUs 941, GPUs 942, FPGAs 943, and accelerators 944 can execute certain instructions that, in combination, can make up the aforementioned computer code. That computer code can be stored in ROM 945 or RAM 946. Transitional data can be also be stored in RAM 946, whereas permanent data can be stored for example, in the mass storage 947 that is internal. Fast storage and retrieve to any of the memory devices can be enabled through the use of cache memory, that can be closely associated with one or more CPU 941, GPU 942, mass storage 947, ROM 945, RAM 946, and the like.

The computer readable media can have computer code thereon for performing various computer-implemented operations. The media and computer code can be those specially designed and constructed for the purposes of the present disclosure, or they can be of the kind well known and available to those having skill in the computer software arts.

As an example and not by way of limitation, the computer system 900 having architecture , and specifically the core 940 can provide functionality as a result of processor(s) (including CPUs, GPUs, FPGA, accelerators, and the like) executing software embodied in one or more tangible, computer-readable media. Such computer-readable media can be media associated with user-accessible mass storage as introduced above, as well as certain storage of the core 940 that are of non-transitory nature, such as core-internal mass storage 947 or ROM 945. The software implementing various embodiments of the present disclosure can be stored in such devices and executed by core 940. A computer-readable medium can include one or more memory devices or chips, according to particular needs. The software can cause the core 940 and specifically the processors therein (including CPU, GPU, FPGA, and the like) to execute particular processes or particular parts of particular processes described herein, including defining data structures stored in RAM 946 and modifying such data structures according to the processes defined by the software. In addition or as an alternative, the computer system can provide functionality as a result of logic hardwired or otherwise embodied in a circuit (for example: accelerator 944), which can operate in place of or together with software to execute particular processes or particular parts of particular processes described herein. Reference to software can encompass logic, and vice versa, where appropriate. Reference to a computer-readable media can encompass a circuit (such as an integrated circuit (IC)) storing software for execution, a circuit embodying logic for execution, or both, where appropriate. The present disclosure encompasses any suitable combination of hardware and software.

While this disclosure has described several non-limiting example embodiments, there are alterations, permutations, and various substitute equivalents, which fall within the scope of the disclosure. It will thus be appreciated that those skilled in the art will be able to devise numerous systems and methods which, although not explicitly shown or described herein, embody the principles of the disclosure and are thus within the spirit and scope thereof.

What is claimed is:

1. A method performed by at least one processor, the method comprising:
obtaining a plurality of videos of a body of a person, the plurality of videos including a first video of the person from a first perspective that is captured by a first camera during a time period, and a second video of the person from a second perspective, different from the first perspective, that is captured by a second camera during the time period;
estimating a three dimensional (3D) pose of the person based on the plurality of videos without depending on any marker on the person, the estimating comprising obtaining a set of 3D body joints;
obtaining an animation of motion of the set of 3D body joints that corresponds to motion of the person during the time period;
performing an analysis of the motion of the set of 3D body joints; and
indicating a rehabilitation evaluation result of the analysis or a rehabilitation training suggestion, based on the analysis, via a display or a speaker.

2. The method of claim 1, wherein
the performing the analysis comprises calculating at least one rehabilitation evaluation indicator based on the motion of the set of 3D body joints.

3. The method of claim 2, wherein
the performing the analysis further comprises selecting the at least one rehabilitation evaluation indicator to be calculated based on an input from a user.

4. The method of claim 1, further comprising:
displaying the animation of the motion of the set of 3D body joints.

5. The method of claim 4, wherein
the animation of the motion of the set of 3D body joints is displayed in real-time with respect to the motion of the person during the time period.

6. The method of claim 5, wherein
the animation includes images of the body of the person combined with the set of 3D body joints.

7. The method of claim 1, wherein
the plurality of videos, that are obtained, further includes a third video of the person from a third perspective, different from the first perspective and the second perspective, that is captured by a third camera during the time period.

8. The method of claim 7, wherein
the first perspective is a left side view of the person, the second perspective is a front view of the person, and the third perspective is a right side view of the person.

9. The method of claim 8, wherein
the second camera captures the second video at a higher height than a height at which the first camera captures the first video and a height at which the third camera captures the third video.

10. The method of claim 9, wherein
the height at which the first camera captures the first video and the height at which the third camera captures the third video are a same height.

11. A system comprising:
a plurality of cameras, the plurality of cameras configured to each obtain a respective video from among a plurality of videos of a body of a person, the plurality of cameras comprising:
a first camera configured to obtain a first video, from among the plurality of videos, of the person from a first perspective during a time period, and
a second camera configured to capture a second video, from among the plurality of videos, of the person from a second perspective, different from the first perspective, during the time period;
a display or a speaker;
at least one processor; and
memory comprising computer code, the computer code comprising:
first code configured to cause the at least one processor to estimate a three dimensional (3D) pose of the person by obtaining a set of 3D body joints, based on the plurality of videos without depending on any marker on the person;
second code configured to cause the at least one processor to obtain an animation of motion of the set of 3D body joints that corresponds to motion of the person during the time period;
third code configured to cause the at least one processor to perform an analysis of the motion of the set of 3D body joints; and
fourth code configured to cause the at least one processor to indicate a rehabilitation evaluation result of the analysis or a rehabilitation training suggestion, based on the analysis, via the display or the speaker.

12. The system of claim 11, wherein
the third code is configured to cause the at least one processor to perform the analysis by calculating at least one rehabilitation evaluation indicator based on the motion of the set of 3D body joints.

13. The system of claim 12, wherein
the third code is further configured to cause the at least one processor to select the at least one rehabilitation evaluation indicator to be calculated based on an input from a user.

14. The system of claim 11, wherein
the system comprises the display, and
the second code is further configured to cause the at least one processor to cause the display to display the animation of the motion of the set of 3D body joints.

15. The system of claim 14, wherein
the second code is configured to cause the at least one processor to cause the display to display the animation in real-time with respect to the motion of the person during the time period.

16. The system of claim 15, wherein
the animation includes images of the body of the person combined with the set of 3D body joints.

17. The system of claim 11, wherein
the plurality of cameras further comprises a third camera that is configured to obtain a third video of the person from a third perspective, different from the first perspective and the second perspective, during the time period.

18. The system of claim 17, wherein
the first perspective is a left side view of the person, the second perspective is a front view of the person, and the third perspective is a right side view of the person.

19. The system of claim 18, wherein
the second camera is at a higher height than a height of the first camera and a height of the third camera.

20. A non-transitory computer-readable medium storing computer code that is configured to, when executed by at least one processor, cause the at least one processor to:
estimate a three dimensional (3D) pose of a person by obtaining a set of 3D body joints based on a plurality of videos of a body of the person without depending on any marker on the person;
obtain an animation of motion of the set of 3D body joints that corresponds to motion of the person during a time period;
perform an analysis of the motion of the set of 3D body joints; and
indicate a rehabilitation evaluation result of the analysis or a rehabilitation training suggestion, based on the analysis, via a display or a speaker,
wherein the plurality of videos include a first video of the person from a first perspective that is captured by a first camera during the time period, and a second video of the person from a second perspective, different from the first perspective, that is captured by a second camera during the time period.
